(19) United States
(12) Patent Application Publication
(10) Pub. No.: US 2025/0281534 A1
(43) Pub. Date: Sep. 11, 2025

(54) METHODS AND COMPOSITIONS FOR IMPROVING IMMUNOTHERAPY

(71) Applicant: Regents of the University of Minnesota, Minneapolis, MN (US)

(72) Inventors: Bruce R. Blazar, Minneapolis, MN (US); Jemma Larson, Minneapolis, MN (US); Michael Loran Dustin, Oxford (GB); Ewaldus Bernardus Compeer, Chesham (GB)

(73) Assignee: Regents of the University of Minnesota, Minneapolis, MN (US)

(21) Appl. No.: 18/631,916

(22) Filed: Apr. 10, 2024

Related U.S. Application Data
(60) Provisional application No. 63/458,327, filed on Apr. 10, 2023.

(51) Int. Cl. A61K 35/17; A61K 39/00; A61P 35/00

(52) U.S. Cl. CPC A61K 35/17; A61K 40/31; A61P 35/00

(57) ABSTRACT

This document describes methods and compositions for producing CD8+ induced Treg (iTreg) cells that include a construct expressing a ligand for use in cell therapy.

### FEDERALLY SPONSORED RESEARCH OR DEVELOPMENT

This invention was made with government support under HL118979 awarded by National Institutes of Health. The government has certain rights in the invention.

### TECHNICAL FIELD

This disclosure generally relates to immunotherapy and, more specifically, methods and compositions for improving immunotherapy.

### BACKGROUND

CD8+ regulatory T cells (Treg) remain a largely understudied bifunctional T-cell subset capable of simultaneous suppressor and cytolytic functions (Bolivar-Wagers et al., 2022, Front. Immunol., 13). CD8+ induced Treg (iTreg) express canonical Treg markers and can secrete immunosuppressive cytokines and cytotoxic molecules, including granzymes and perforin. Infusion of ex vivo-generated murine CD8+ iTreg lessens acute graft-vs-host disease (GVHD), although less than ex vivo-generated murine CD4+ iTreg, but the CD8+ iTregs are superior at preserving murine graft-vs-tumor (GVT) activity.

### SUMMARY

Methods and compositions for producing human CD8+ iTreg cells that include a construct expressing a ligand are described herein. Such cells can be used in a variety of cell therapy applications.

In one aspect, methods of increasing the cytolytic and/or anti-tumor function of CD8 iTregs are provided. Such methods typically include transducing the CD8 iTregs with a construct, wherein the construct expresses a ligand, thereby producing ligand-expressing CD8 iTregs, thereby increasing the cytolytic and/or anti-tumor function of the ligand-expressing CD8 iTregs compared to CD8 iTregs not containing or expressing the ligand. In some embodiments, the transduction does not abrogate suppressor function of the CD8 iTregs.

In some embodiments, the construct expressing a ligand is a CAR construct. In some embodiments, the construct expresses a ligand selected from CD19, CD33, CD123, CD45, CD83, and VISTA. In some embodiments, the ligand expressed by the construct is a ligand that specifically recognizes a pathogenic antigen, a tumor antigen, a foreign antigen, or a self-antigen.

In some embodiments, the ligand-expressing iTreg exhibit cytotoxicity and suppressor function. In some embodiments, the ligand-expressing iTregs are cytotoxic to tumor cells.

In another aspect, methods of delivering therapy to a patient in need thereof is provided. Such methods typically include providing ligand-expressing CD8 iTregs; introducing the ligand-expressing CD8 iTregs into the patient in need thereof, thereby delivering therapy to a patient in need thereof.

In some embodiments, the CD8 Tregs used to produce the ligand-expressing CD8 iTregs are obtained from the patient.

In some embodiments, the method does not suppress graft-vs-tumor (GVT) activity in the patient. In some embodiments, the method augments graft-vs-tumor (GVT) activity in the patient.

In some embodiments, the method reduces or eliminates tumor cells in the patient. In some embodiments, the patient has undergone a hematopoietic stem cell transplantation (HSCT).

In one aspect, methods of making ligand-expressing iTreg cells are provided. Such methods typically include providing CD8+ T cells from an individual; introducing a construct expressing a ligand into the CD8+ T cells to produce ligand-expressing T cells; and culturing the ligand-expressing T cells under conditions in which ligand-expressing induced T regulatory (iTreg) cells are produced, thereby making ligand-expressing iTreg cells.

In some embodiments, the CD8+ T cells are provided in peripheral blood.

In some embodiments, the construct expressing a ligand is introduced into the CD8+ T cells using transduction. In some embodiments, the construct expressing a ligand is a CAR construct. In some embodiments, the ligand expressed by the construct is CD19. In some embodiments, the ligand expressed by the construct is a ligand that specifically recognizes a pathogenic antigen, a tumor antigen, a foreign antigen, or a self-antigen.

In some embodiments, the conditions under which ligand-expressing iTreg cells are produced comprises culturing the ligand-expressing T cells in the presence of IL-2, TGF-beta and rapamycin. In some embodiments, the conditions under which ligand-expressing iTreg cells are produced comprises culturing the ligand-expressing T cells in the presence of retinoid acid, vitamin C, vitamin D3, indoleamine 2,3 dioxygenase, tolerigeneic dendritic cells, antigen-presenting cells, or combinations thereof.

In some embodiments, the ligand-expressing iTreg cells are CD103+, CD39+, and Foxp3+. In some embodiments, the ligand-expressing iTreg cells exhibit cytotoxicity and suppressor function.

In another aspect, methods of inhibiting, preventing and/or treating GVHD while maintaining and/or enhancing GVT activity are provided. Such methods typically include delivering ligand-expressing iTreg cells to a patient in need thereof, thereby inhibiting or preventing GVHD while maintaining GVT activity in the patient.

In some embodiments, the patient has undergone a hematopoietic stem cell transplantation (HSCT). In some embodiments, the ligand-expressing iTreg cells are made by the methods described herein.

In still another aspect, methods of reducing tumor burden and/or delaying tumor-related mortality in a patient are provided. Such methods typically include delivering ligand-expressing iTreg cells to a patient in need thereof, thereby reducing tumor burden and/or delaying tumor-related mortality in the patient. In some embodiments, the ligand-expressing iTreg cells are made by the methods described herein.

In some embodiments, the method does not suppress graft-vs-tumor (GVT) activity in the patient. In some embodiments, the method augments graft-vs tumor (GVT) activity in the patient. In some embodiments, the method reduces or eliminates tumor cells in the patient.

In one aspect, methods of controlling an adverse immune response in a patient are provided. Such methods typically include delivering ligand-expressing iTreg cells to a patient in need thereof, thereby controlling the adverse immune response in the patient. In some embodiments, the ligand-expressing iTreg cells are made by the methods described herein.

In some embodiments, the patient has undergone a hematopoietic stem cell transplantation (HSCT). In some embodiments, the adverse immune response is graft-vs-host disease (GVHD), an autoimmune disease, organ grafting, overly robust anti-pathogen responses. In some embodiments, the method does not suppress graft-vs-tumor (GVT) activity in the patient. In some embodiments, the method augments graft-vs-tumor (GVT) activity in the patient. In some embodiments, the method reduces or eliminates tumor cells in the patient.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 shows that human CD8+ iTreg cells express Foxp3, CD39, and high levels of CD103+ compared to conventional CD8+ cytotoxic T cells (CTLs).

FIG. 2 are graphs showing that human CD8+ iTreg are highly suppressive and cytolytic. PB CD8+ iTreg similarly suppress as CD4+ iTreg replicated in 5 studies with different donors. S.D is shown (top). CD8+ iTreg and CD8+ CTLs have equal killing of primary B cell leukemia (Nalm-6) tumor cells (effector:target (E:T)=5:1; 48 hr) with an &CD3×&CD19 bispecific engager (blinatumomab (BLIN)) (bottom).

FIG. 3 shows that, following 5 days of stimulation with αCD3×αCD28 Dynabeads, CD8+CD103+ iTreg are enriched for a highly potent granzyme B (GzmB) hi granulysin (GNLY) hi CD8+ population, demonstrating that CD103+CD8+ iTreg have a highly potent cytotoxic profile compared to CD8+ CTLs.

FIG. 4 shows that human CD8+ iTreg cells suppress xenogeneic (xeno) GVHD. Survival of irradiated immune deficient NOD.Cg-Prkdc scid Il2rg tm1Wjl/SzJ (NSG) recipients (5/group) given human peripheral blood mononuclear cells (PBMCs) (2.5 M)±hCD4+ CD4+ thymic Treg (tTreg), or hCD8+ CTLs, or hCD8+CD103+ iTreg (4 M), or no Tregs. hCD8+ iTreg vs hCD8+ CTLs, or hCD4+ tTreg (P<0.0018).

FIG. 5 are graphs showing that in vitro destabilization of FOXP3 expression in CD8+ iTreg did not correlate with loss in suppressor function. CD8+ iTreg were cultured for 48 hr under control or inflammatory conditions (low dose IL-2+/−IL-1 and IL-6). CD8+ iTreg purity was quantified by flow cytometry (left). Suppressor function was ascertained via in vitro suppression assay (1:8 Treg:PBMC ratio; right) Replicated with 2 different donors. S.D. is shown.

FIG. 6 shows that CD103⁺ CD8+ iTreg are more suppressive in vitro. CD8+ iTreg cultures were column sorted into CD103+ and CD103^{neg} sub-populations. Suppressor function of CD103⁺ vs. CD103^{neg} iTreg was compared to unseparated (UnSep) CD8+ iTreg and CD8+ CTLs. Replicated in 2 assays with different donors. S.D is shown.

FIG. 7 shows that over expression of Foxp3 alone in CD8+ T-cells does not induce suppressor function compared to CD8+ iTreg. CD8+CD25neg T cells were transduced with lentivirus encoding Foxp3. Following transduction, Foxp3+ Tc were cultured/expanded for 2-weeks with IL-2 and CD3/28 stimulation. Donor-matched T-cells without editing were cultured under iTreg conditions for 2-weeks. On day 14, cells were analyzed by flow and for in vitro suppressor function. Foxp3-transduced T cells had increase Foxp3 expression, but low expression of CD103/CD39, compared to CD8+ iTreg. Foxp3-transduced T cells did not gain potent suppressor function compared to donor matched CD8+ iTreg.

FIG. 8A-8F show that generation of CAR19-41BB CD9 iTreg amplifies antigen specific cytotoxicity without compromising T-cell suppression. Following transduction with lentivirus encoding chimeric antigen receptor (CAR) anti-CD19scFV/4-1BB-EGFR and isolation of cells expressing truncated epidermal growth factor receptor (EGFR), CAR19 T cells were cultured under iTreg conditions to generate CAR19 CD8+ iTreg, or with IL-2 alone to generate CAR19 CTLs. (8A) CD8 iTreg purity following transduction and subsequent CD8 iTreg induction protocol. (8B) CAR19-41BB transduction efficiency following CD8 iTreg generation. CAR19 CD8+ iTreg were generated with high iTreg purity and >90% EGFR expression. (8C) Addition of CAR19 did not compromise CD8+ iTreg suppressive function. 72 hr in vitro suppression assay demonstrating suppressive capacity of CD8 iTreg expressing CAR19-41BB compared to Mock control CD8 iTreg and donor matched CAR-41BB and mock CTLs. (8D) 48 hr in vitro killing assay demonstrating ability of CD8 iTreg expressing CAR19-41BB, compared to mock control, to kill Nalm-6 tumor cells at 5:1 ratio (iTreg to tumor cells). (8E) Incucyte time-lapse quantification of in vitro tumor killing of CD19+ Nalm-6 tumor cells by mock or CAR19-41BB CD8+ CTLs and CD8 iTreg during 72 hr co-culture. (8F) Incucyte time-lapse quantification of in vitro tumor killing of CD19-KO Nalm-6 tumor cells by mock or CAR19-41BB CD8+ CTLs and CD8 iTreg during 72 hr co-culture.

FIG. 9 shows flow cytometry-based killing assay demonstrating that human CAR19/4-1BB CD8+ iTreg and human CAR19 CTLs have equal capacity to kill CD19+ Nalm-6 tumor cells (left), with no effect of CD19-knockout (KO) Nalm-6 targets (right), and no bystander killing of non-antigen specific targets (bottom) in mixed target assays. Replicated in 3 studies with different donors. S.D. is shown.

FIG. 10 shows that human CAR19/4-1BB CD8+ iTreg have superior clearance of CD19+ Nalm-6 leukemia cells in NSG mice compared to CAR19/4-1BB conventional CTLs. Mice were given GFP/luciferase transduced Nalm-6 (1e6); 7 days later, mice received tumor only, CAR19-4-1BB, mock CD8+ iTreg, or conventional CTLs (IL-2 given D7-D28). Shown are representative time points of weekly bioluminescent imaging (BLI) at the indicated times (10A), quantification of Nalm-6-Luc tumor burden (10B), and survival (10C) (n=5/group).

FIG. 11 shows that human CAR19/4-1BB CD8+ iTreg have greater protection against CD19+ Nalm-6 leukemia compared to CAR19/CD28+CD8+ iTreg in vivo. Shown is GVL survival (top left) (n=5/group). No difference in cytolytic potency was observed in vitro after 48 hrs (top right). Following tumor 2nd consecutive tumor challenge, CAR19/CD28 CD8+ iTreg had significantly reduced tumoricidal activity compared to CAR18/4-1BB CD8+ iTreg, as shown in Incucyte time-lapse quantification of in vitro tumor killing (bottom left).

FIG. 12A-F shows CD8+ iTreg release supramolecular attach particles (SMAPs). FIG. 12A shows a schematic of the experiment. FIG. 12B shows GZMB (red) and perforin (PRF1) (green) IF staining without flushing cells. FIGS. 12C and 12D show that, after flushing cells, GZMB, PRF1 and wheat germ agglutinin (WGA) (blue) particles were attached to SLB. *GZMB+/WGA+ SMAPs. FIG. 12E shows the quantification of GZMB particles with different cell types and bilayer compositions. FIG. 12F shows GZMB and PRF1 co-localization in CTL and CAR19 CD8+ iTreg is positive and not significantly (ns) different. Mann-Whitney: *=p<0.05; ***P<0.0001).

FIG. 13 shows CD103+CD8+ iTregs are highly cytotoxic and rely less on soley Granzyme B and require Thsb4 expression for cellular and SMAP-mediated killing of NALM-5 leukemia cells. Anti-leukemic cell killing by CD103+CD8+ iTreg was assessed upon different inhibitor treatments. CD8 (FIGS. 13A and 13B) Treg and CTLs rely on perforin for CD3-CD19 bi-specific engager (BLIN or BiTE, respectively)-induced killing of Nalm-6 leukemia cells, CD8 iTreg cytotoxicity was less affected by GzmB-specific inhibition but had similar sensitivity to pan-Granszyme protease inhibitors as CTLs in short-term caspase killing assay (FIG. 13B). Unique from CD8 CTLs, CD8 iTreg release thbs4+ SMAPs partials, (13C and 13D) which are required for effective tumoricidal activity in vitro (13E).

FIG. 14A-14E shows that CAR19-41BB CD8 suppress simultaneously suppress GVHD and reduce tumor burden in a xenogeneic GVHD residual tumor model. Treatment with CAR19+ CTLs produced robust antileukemic activity protecting mice from fatal tumor progression (14B-14D). However, CD19+ CAR CTL recipients rendered no protection against GVHD, resulting in significant early weight loss in those animals (14E) and only a marginal improvement in median survival compared to Nalm-6/PBMC recipients (29-vs. 20-days, respectively; p<0.0026; 14B). Conversely, mice receiving CAR19 iTreg intervention had reduced early weight loss (14E), blunted leukemia progression (14C and 14D), and significantly prolonged animal survival compared to CAR19 CTL recipients (43-day median survival; p<0.0173; 14B).

FIG. 15 shows the identification of CD103+ Thbs4hiGZmMhi CD8+ iTregs releasing Thbs4+ SupraMolecular Attach Particles (SMAPs).

### DETAILED DESCRIPTION

CAR19 T-cells can induce complete remission with varying efficacy and durability in adult B cell acute lymphocytic leukemia patients. However, the frequency and severity of cytokine release syndrome and neurotoxicity are major barriers for CAR T cell therapies. Unlike conventional CAR T cells, Treg cells suppress inflammatory reactions, and are effective in suppressing graft-vs-host disease (GVHD). We previously reported that mouse CAR19/4-1BB CD4+ tTreg cells reduced GVHD and maintained graft-vs-tumor (GVT) efficacy against hCD19+ cells, however, as described herein, preliminary data unexpectedly suggests that human CD8+ iTreg may be superior suppressors of xenoGVHD, compared to CD4+ tTreg, at low Treg:PBMC ratios. Specifically, the studies described herein demonstrate that CAR19/4-1BB human CD8+ iTreg maintain suppressor function and significantly improved clearance of CD19+ Nalm-6 tumor cells in vivo, compared to CAR19/4-1BB T cells, and exhibited reduced tumor-related mortality in a xenoGVHD model.

Clinical translation of Treg cell therapy has been hampered by variability in Treg potency and high dosing requirements. CD8+ iTregs are readily generated in high numbers from highly abundant CD8+ T cells, as contrasted to the rare CD4+ Tregs, solving a production problem. Moreover, current clinical therapies are largely limited in their ability to either target and eliminate malignancies, or suppress inflammatory and allogenic immune responses in vivo. Preclinical and clinical studies show that adoptive transfer of CD4+ Treg can be effective for preventing GVHD, however, there is the potential for loss of GVT activity with standard CD4+ Treg therapies, leading to increased risk of relapse. Adoptive CAR T cell therapies have drastically advanced the treatment of hematological malignancies in the clinical setting with potent anti-tumor activity; however, CAR T therapies are associated with significant toxicity and inflammation, and can further exacerbate GVHD severity.

Recently, we have shown that CD4+ Tregs expressing anti-CD19scFv (CAR19) can be an effective strategy to suppress GVHD without loss of GVT activity and with reduced risk of CAR T-associated toxicity. Similarly, CAR CD8+ iTreg therapies may offer the opportunity for a dual function cell therapy. CAR CD8+ iTreg can simultaneously suppress pathogenic allogeneic immune responses following HSCT and actively engage in anti-tumor activity in vivo. While pre-clinical CAR CD4+ Treg studies have shown similar action, we suggest that the technically far more simple large-scale generation of CD8+ iTreg products, along with the active anti-tumor activity observed with CAR CD8+ iTreg cells, are both distinct advantages over other previous CAR Treg therapies. T cell receptor (TCR)-transduced Tregs would have similar benefits and could target intracellular antigens and cell surface antigens for which antibodies are not available to generate CAR. TCRs can be selected having various affinities for optimal potency. With dual cytolytic and suppressive capacity, the best of both worlds of direct cytolytic activity and suppressing adverse immune responses including overly robust antitumor/pathogen/autoimmune responses can be achieved.

As described in more detail below, CD8+ induced Treg (iTreg) cell therapy expressing αCD19scFv (CAR19) were generated. Human peripheral blood CD8+ T-cells were first transduced with a viral vector encoding the target chimeric antigen receptor (CAR) of interest, and subsequently differentiated into CD8+ iTreg by culturing transduced T-cells with IL-2, TGF-beta and rapamycin. Generated CD8+ iTreg have high expression of CD103, CD39 and Foxp3, retain the inherent cytotoxicity of their CD8+ cytotoxic lymphocyte (CTL) counterparts, and have potent suppressor function; are able to significantly delay mortality in a xenogenic model of GVHD (p=0.0018). In a comparative 48 hr Incucyte continuous killing assay against CD19+ Nalm-6 targets, using CAR19 CD8+ iTreg and CAR19 CTLs generated from the same donors, no significant differences were noted; CD8+ iTreg killing was perforin-dependent. No killing was observed against CD19-KO Nalm-6 targets. Unexpectedly, CAR19 CD8+ iTreg were found to be significantly more effective at limiting tumor burden in vivo and significantly delayed tumor-related mortality in a xenogeneic Nalm-6 tumor model compared to CAR19 CTLs (p=0.0018). Without being bound by theory, preliminary data suggest that this may be the result of unique homing and persistence properties of CD8+ iTreg (compared to CD8+ CTLs) and of a prolonged secretion of cytolytic molecules including perforin, granulysin, granzymes A, B and M, TNF-alpha, and interferon-gamma as secreted proteins or in the form of SMAPs, as described herein, or exosomes. Taken together, the preliminary data provide a rationale for using transduced CD8+ iTreg-based therapies to simultaneously prevent GVHD and promote anti-tumor activity.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Methods

Suppression assay. Frozen stock of human PBMCs were thawed, rested overnight and stained with 2.5 μM carboxyfluorescein succinimidyl ester (CFSE) (Life technologies, Thermo Fisher Scientific). Stained PBMCs were then mixed with CD8 iTreg at Treg/PBMC ratios of 0:1, 1:2, 1:4, 1:8 and 1:16 in the presence of anti-CD3/CD28 Dynabeads (2:1 bead to PBMC ratio). PBMC proliferation was analyzed after 72 hours in culture.

Killing assay. CD19+ or CD19-KO Nalm-6-GFP/firefly-luciferase (luc) tumor cells were cultures in vitro. Tumor cells were mixed with CD8 iTreg at 5:1 T-cell to tumor ratio. Killing assays without CAR19 utilized an anti-CD3×anti-CD19 bispecific engager (BLIN), which was included in the cultures at 25 ng/mL. Tumor killing was analyzed after 48 hours in culture.

XenoGVHD. PBMCs (2.5e6) were given to irradiated NSG mice to induce GVHD. Groups (n=5/group) include: 1, PBMC only; 2, CD8 iTreg; 3, CD8 CTL; and 4, CD4 tTreg. Readouts: Survival daily, clinical scores and weights 2×/week, with d10 flow analysis for detailing frequency and phenotype of T-cell populations (human CD45, CD4, CD8 iTreg).

GVT. Irradiated NSG mice received Nalm-6-GFP/firefly-luciferase (luc) tumor cells (1 M) d0 followed by T-cells (10 M) on d7. Groups (n=5/group) include: 1, Tumor only; 2, 3, Mock vs. CAR19/4-1BB CD8 iTreg; 4, 5, Mock vs. CAR19/4-1BB CD8 CTL; or 6, CAR19/C28 CD8 iTreg. Readouts: Survival was monitored daily, and twice weekly clinical scores and body weight, and tumor progression using sequential total body BLI of Nalm-6-GFP/luc tumor cells after intraperitoneal D-luciferin (Perkin Elmer Inc.) detected on an IVIS Lumina II imaging system and analyzed by Living Image 4.5 software.

### Example 2—Preliminary Data

(1) Generation of bifunctional human CD8 iTreg. We previously reported that CD4⁺ iTreg can be generated on a large scale from human PB CD4⁺CD25^{neg} T-cells, which suppressed disease in a xenoGVHD model (Hippen et al., 2011, Amer. J. Transplant., 11 (6): 1148-57). We adapted our CD4 iTreg protocol to generate CD8 iTreg from CD8⁺CD25^{neg} T-cells. With several distinct donors, we generated ˜75% of CD4 iTreg numbers.

Protocol to generate iTregs: CD8+CD25neg T-cells were isolated from human PBMCs and stimulated in vivo with irradiated KT64/86 cells loaded with anti-CD3 (1:1 T-cell to KT cell). Cells were resuspended at 0.25×10⁶/ml (T-cells) in x-vivo media, supplemented with 300 U/mL IL-2, 9 ng/ml TGFβ, and 109 nM rapamycin, cultures were incubated at 37° C. for 7 days (splitting culture every 2-3 days to maintain cell density >1×10⁶/mL). Culture was re-stimulated at day 7, as described above. Following the 2^{nd} stimulation (d14), CD8 iTreg were harvested, counted, and used for in vitro and in vivo studies.

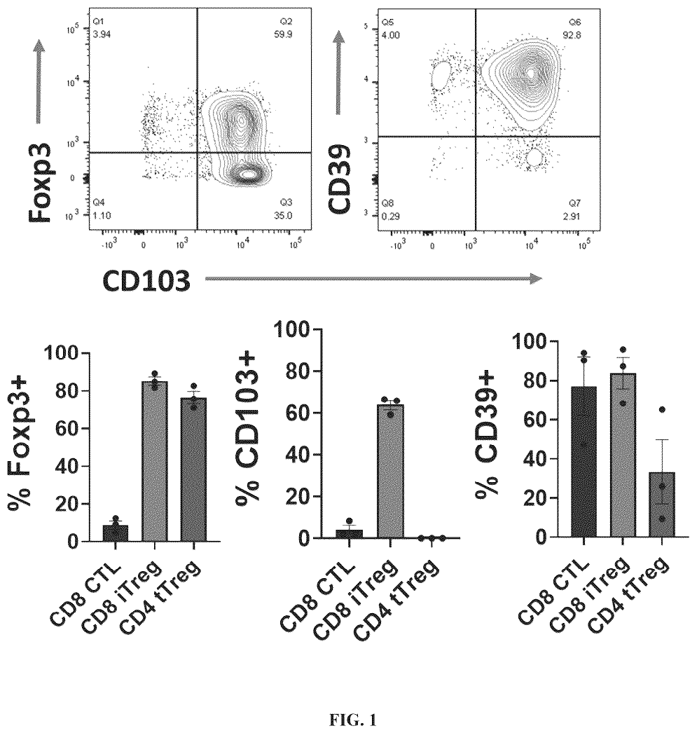

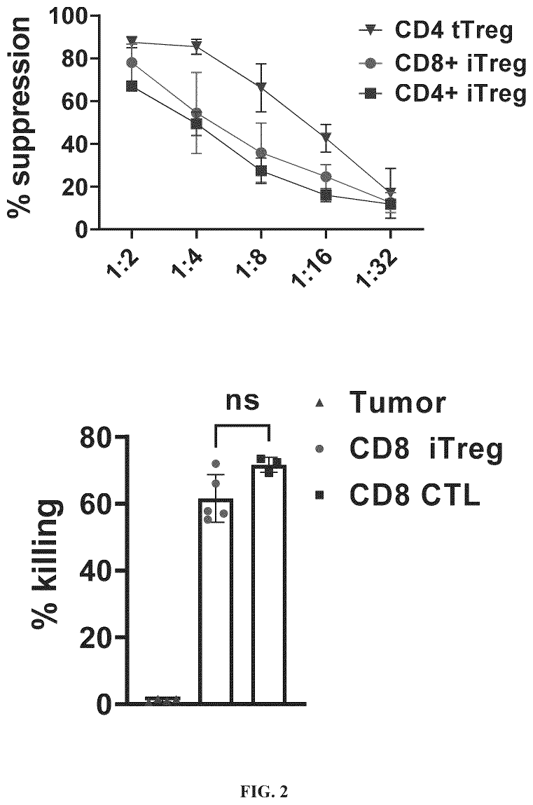

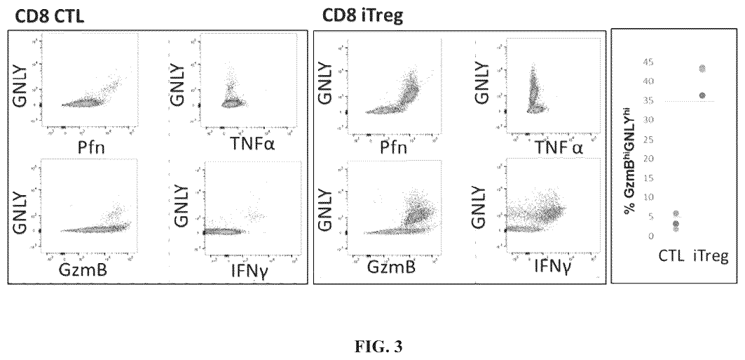

CD8 iTreg expressed canonical Treg markers, FoxP3 and CD25, and high levels of CD103 and CD39 compared to CD8 CTLs (FIG. 1), with similar suppressor potency as CD4 iTreg (FIG. 2, top). In vitro Nalm-6 killing capacity of CD8 iTreg was similar to conventional CD8 CTLs with an αCD3×αCD19 bispecific engager (BLIN) (FIG. 2, bottom). In vitro CD3/28 stimulation enriched for a highly cytotoxic subset in CD8 iTreg, but not CD8 CTLs. 35-42% of CD8 iTreg, compared to 3-7% of CD8 CTLs, co-expressed granzyme B (GzmB), granulysin (GNLY), INFγ and TNFα (FIG. 3).

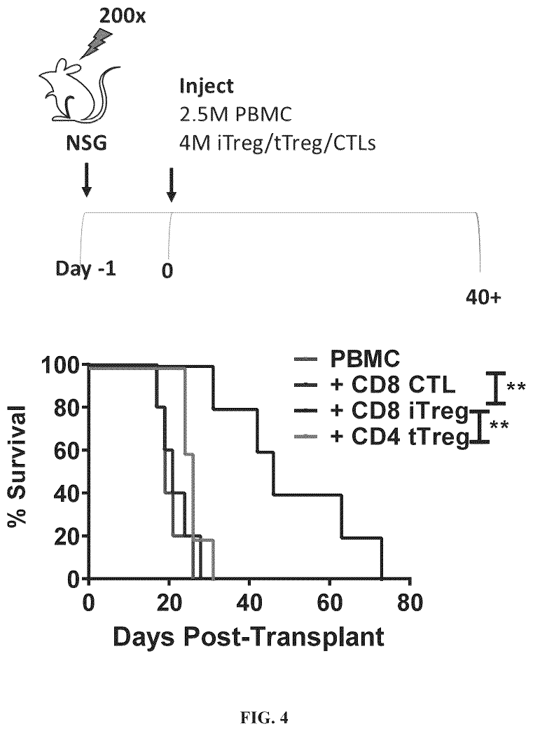

(2) CD8 iTreg suppressed GVHD. In murine models of GVHD, in vivo- and ex vivo-generated CD8 iTreg have been shown to be potent suppressors of GVHD, while preserving GVT activity. We showed that ex vivo-generated human CD8 iTreg suppressed xenoGVHD at a reduced Treg:PBMC ratio (FIG. 4). Irradiated NSG mice (n=5/group) were administered with human PBMCs (2.5 M) and either CD8 iTreg, CD8 CTLs, or no T-cells (4 M) on day 0 (d0). Mice receiving CD8 iTreg had significantly improved survival, and reduced weight loss and clinical scores compared to CD8 CTLs and no Treg recipients.

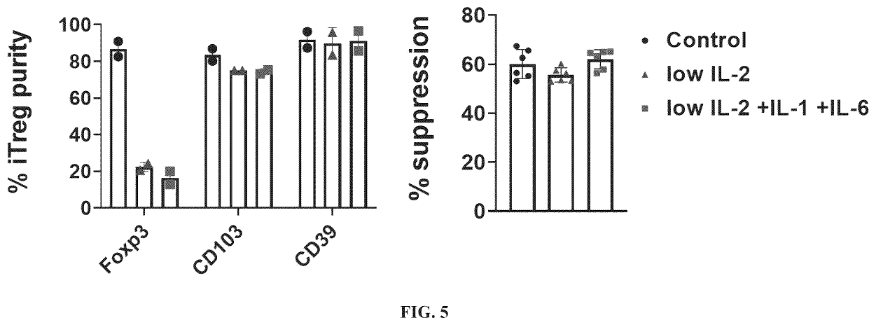

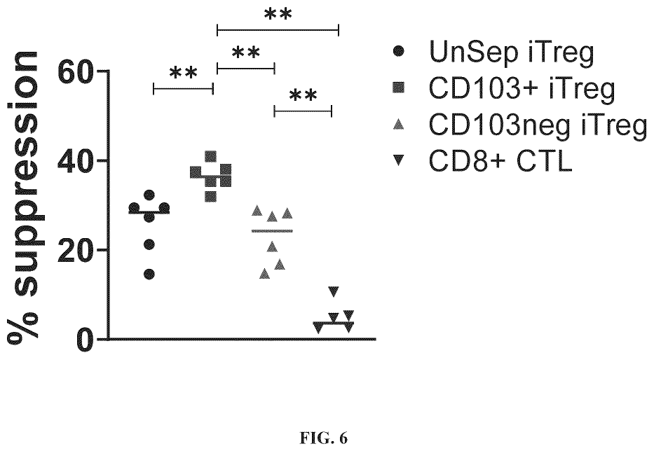

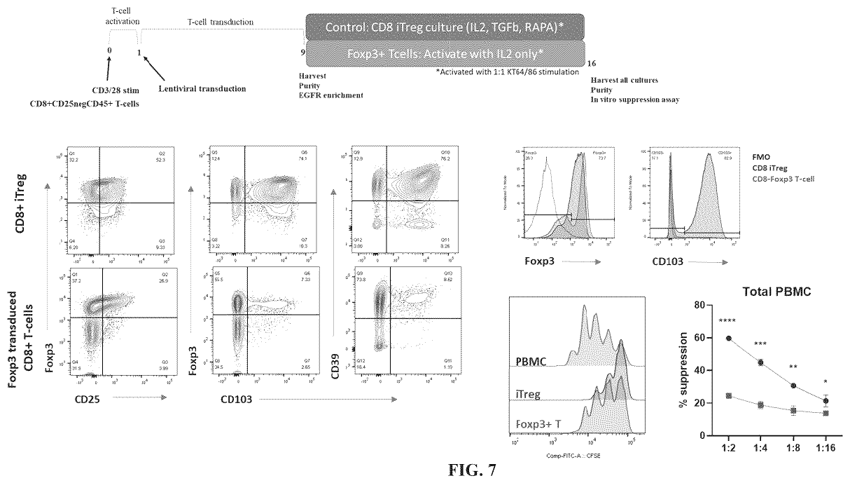

(3) CD8 iTreg stability and suppressor function. For CD4 tTreg and iTreg, Treg suppressor function is closely associated with the constitutive expression of FOXP3. Therefore, the increased instability of FOXP3 expression in CD4 iTreg under inflammatory condition is a major potential weakness of iTreg-based therapies in the clinical setting. Conversely, we showed that the loss of FOXP3⁺CD25⁺ expression in human CD8 iTreg did not correlate with a loss of suppressor function in vitro (FIG. 5). Destabilization of FOXP3 expression was achieved by culturing human CD8 iTreg (Foxp3⁺CD25⁺%>80%) for 48 hrs with low dose IL-2±IL-1 and IL-6. Under pro-inflammatory conditions, CD8 iTreg lost expression of Foxp3/CD25 (<20%) but retained high expression of CD39 (>80%) and CD103 (>75%). Suppressor function was found to correlate with high expression of CD103 (FIG. 6), although both CD103⁺ and CD103^{neg} CD8 iTreg were more suppressive than conventional CD8 CTLs. Similarly, it has previously been reported that murine TGFβ supported CD8⁺CD103⁺ iTreg that suppressed T-cell responses regardless of FOXP3 expression. Transduction of CD8+CD25neg T-cells with lentivirus-encoding Foxp3 alone did not impart a suppressive phenotype or function (FIG. 7). Together, these data suggest that CD103, rather than FOXP3, is central for CD8 iTreg suppressor function; and that CD103 expression is a hallmark of a highly functional memory-like CD8 iTreg population and is required for targeted migration and suppression of xenoGVHD.

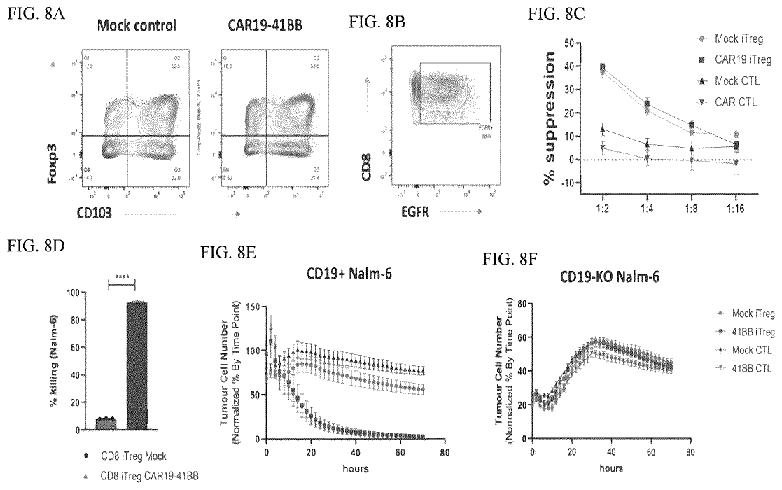

(4) CAR19 hCD8 iTreg. We previously reported that adoptive transfer of murine CAR19 CD4 tTreg suppressed GVHD without loss of GVT and reduced risk of CRS. Subsequently, we generated CD8 iTreg expressing αCD19scFv (CAR19), with no loss in suppressor function (FIG. 8). FIG. 8A shows the extent of CD8 iTreg purity following transduction and subsequent CD8 iTreg induction protocol. FIG. 8B shows the CAR19-41BB transduction efficiency following CD8 iTreg generation. 72 hr in vitro suppression assays demonstrated the suppressive capacity of CD8 iTreg expressing CAR19-41BB compared to mock control CD8 iTreg and donor matched CAR-41BB and mock CTLs (FIG. 8C). 48 hr in vitro killing assay demonstrated the ability of CD8 iTreg expressing CAR19-41BB, compared to mock control, to kill Nalm-6 tumor cells at 5:1 ratio (iTreg to tumor cells) (FIG. 8D). Incucyte time-lapse quantification of in vitro tumor killing of CD19+ (FIG. 2E) or CD19-KO (FIG. 8F) Nalm-6 tumor cells by mock or CAR19-41BB CD8+ CTLs and CD8 iTreg during 72 hr co-culture was captured.

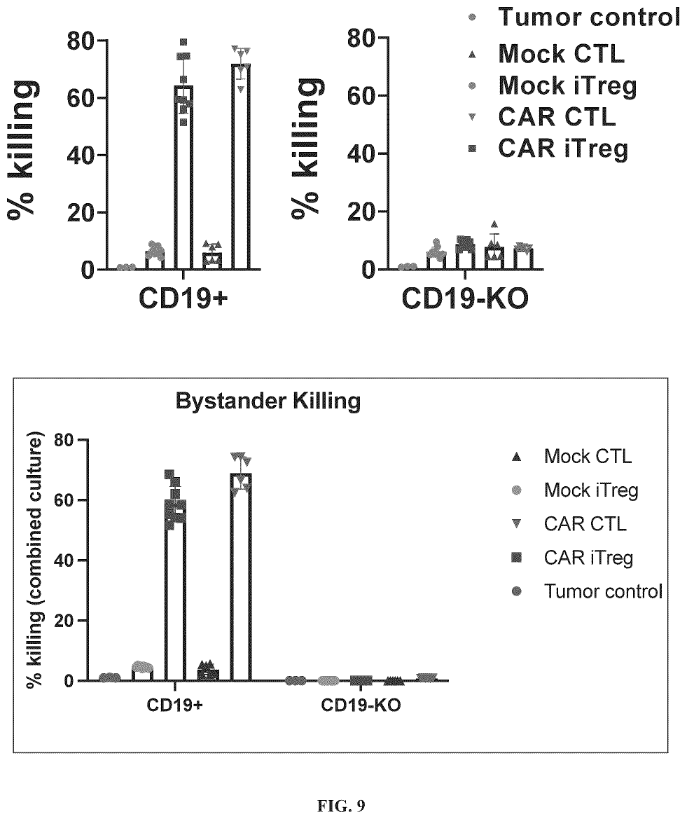

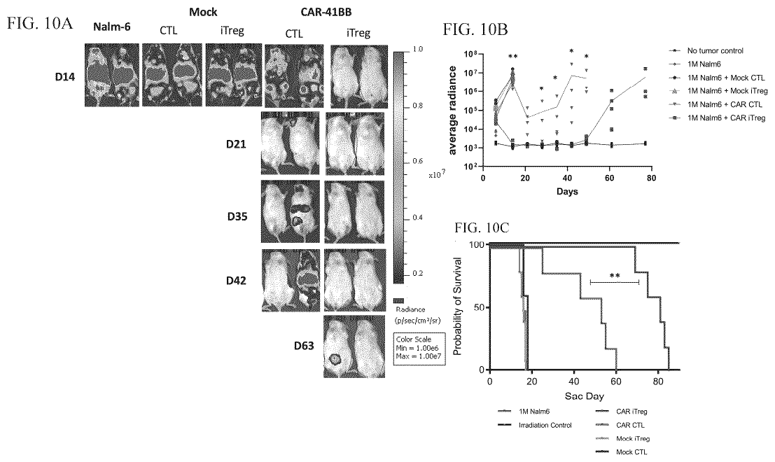

In a 48 hr killing assay against CD19⁺ Nalm-6 targets, using CAR19 CD8 iTreg and CAR19 CTLs generated from the same donors, no significant differences were noted (FIG. 9, left); CD8 iTreg killing was perforin-dependent and granzyme-independent. No killing was observed against CD19-ko Nalm-6 targets (FIG. 9, right), and no evidence of bystander killing was detected in mixed target killing assays (FIG. 9, bottom). These data were replicated using continuous Incucyte killing assays (FIGS. 8E and 8F). Unexpectedly, CAR19 CD8 iTreg were found to be significantly more effective at limiting tumor burden in vivo and significantly delayed tumor-related mortality in a xenogeneic Nalm-6 tumor model compared to CAR19 CTLs (FIG. 10). BLI was performed in irradiated NGS mice (n=5/group) given CD19⁺ Nalm-6-GFP/firefly-luciferase (luc) tumor cells (1 M) d0 and subsequently infused with non-transduced (Mock) or CAR19/4-1BB CD8 iTreg or CD8 CTLs (10 M), or no T-cells on day 7. All mice receiving Mock CD8 iTreg, Mock CD8 CTLs, or no Treg died in <20 days. Mice given CAR19/4-1BB CD8 iTreg had no detectible BLI signal between d14-d42; all mice given CAR19/4-1BB CD8 CTLs had detectable BLI signals from d14 until terminal endpoint. Median survival for CAR19/4-1BB CD8 iTreg recipients was 83 d vs. 53 d for CAR19/4-1BB CTL recipients (p=0.0018). With >90% CAR19 CD8 iTreg purity, these data strongly indicate that human CAR19/4-1BB CD8 iTreg can kill CD19⁺ lymphoma cells, and suggest that CAR19 CD8 iTreg have selective advantage with superior in vivo clearance of antigen bearing tumor cells.

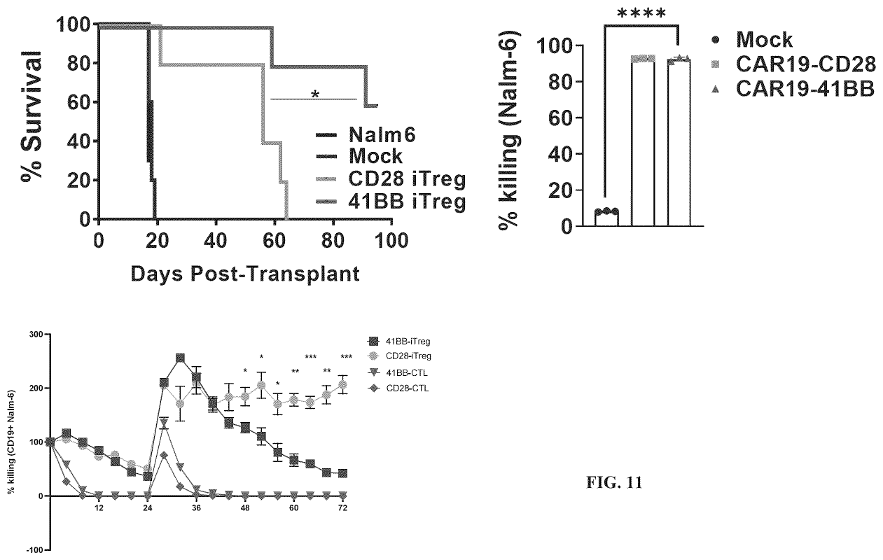

(5) CD8 iTreg CAR19 costimulatory domains. We show that CAR19 CD8 iTreg expressing 4-1BB rather than CD28 co-stimulatory domains are more effective at restricting CD19⁺ Nalm-6 tumor growth in vivo, thereby reducing tumor related mortality (FIG. 11, left), despite no detectible difference in Nalm-6 tumor killing in vitro after 48 hrs (FIG. 11, right). However, following a consecutive tumor rechallenge CAR19 CD8 iTreg expressing CD28 rather than 41BB co-stimulatory domain had significantly reduced tumoricidal activity in vitro (FIG. 11, bottom). Irradiated NGS mice (n=5/group) given CD19⁺ Nalm-6-GFP/luc tumor cells (1 M) d0 with no-Treg or Mock, CAR19/4-1BB or CAR19/CD28 iTreg on d7 (10 M). All mice receiving Mock CD8 iTreg, Mock CD8 CTLs, or no Treg died in <20 days. All mice receiving CAR19/CD28 CD8 iTreg died <63 d, while 3/5 mice receiving CAR/4-1BB CD8 iTreg survived >100 d post-tumor (p=0.0132). It remains unclear the mechanism by which 4/1BB expression augments anti-tumor activity in vivo. Without being bound by theory, we hypothesize that metabolic reprograming resulting from CAR19/4-1BB expression augments the stability and persistence of human CD8 iTreg in vivo and alters the migration of CAR19 CD8 iTreg, thereby further enhancing anti-tumor potency in vivo.

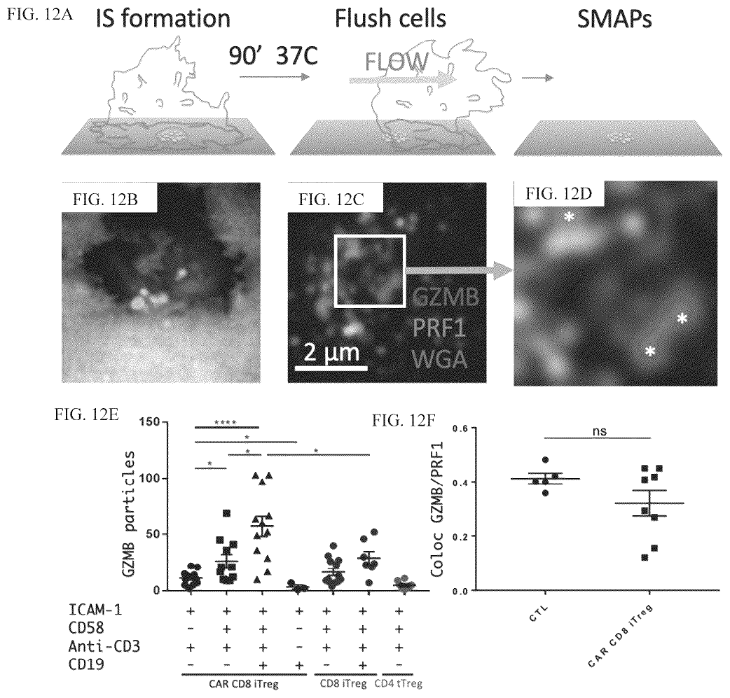

(6) CD8 iTreg SMAPs. We explored CAR19 Treg subset SMAP release using CAR 19 CD8 CTLs as a control. T cells interacted for 90′ with the SLB; GZMB, PRF1 and WGA staining were analyzed by TRIF microscopy±T cell removal to expose putative released SMAPs. ICAM-1, CD58, anti-CD3, and CD19 in different combinations were tested prior to GZMB, PRF1 and WGA TIRF microscopy; T cell removal exposed putative SMAPs released into the synaptic cleft (FIG. 12A). Cell free SLB was stained with anti-GZMB, anti-PRF1 and WGA (binds SMAP shell glycans). Highest SMAP release was CAR19 CD8 iTreg on ICAM-1, CD58, anti-CD3+CD19 proteins (FIG. 12B-E). SMAPs were defined as GZMB+WGA+ particles with significant co-localization GZMB and PRF1 (FIG. 12F) and non-significant trend toward lower PRF1 co-localization in CD8 iTreg vs CD8 CTL. For maximal CD8 iTreg SMAP release, anti-CD3+CD19 engagement was required, suggesting differentiation may have altered activation threshold. Such AND gate required endogenous TCR signals to enable CAR function, potentially reducing toxicity. Adding CD58 significantly increased SMAP release leading to studies of CD2 in Treg killing.

Although CD4 tTreg contained GZMB and PRF1 by confocal imaging, these compartments did not polarize to the IS (not shown) or release SMAP in response to SLB ICAM-1+aCD3, even though these stimuli led to compartment polarization and degranulation from CD8 iTreg. CD8 iTreg not expressing CAR19 didn't show a significant increase in SMAP release by ICAM-1, CD58, anti-CD3+CD19 (FIG. 12E). These data confirmed the importance of the CAR19 responses to SLB CD19 and that CD8 iTreg SMAP release can be enhanced but not unilaterally triggered by CAR19. CD4 tTreg released WGA⁺ particles that lack GZMB and PRF1 (not shown). WGA detects both extracellular vesicles and SMAP-like core-shell glycoprotein particles. These results will enable collection and analysis of CD8 iTreg SMAPs to address their role in serial killing. We will determine if CD4 tTreg have intracellular SMAPs and identify conditions under which they are released into the IS (e.g., through CAR19 expression and exposure to CD19⁺ targets).

### Example 3—Further Characterization of iTreg Cells

Our characterization of hCD8 iTreg was extended and we demonstrated that CD8 iTreg are enriched for a highly cytotoxic subpopulation unique to cytotoxic CD8+ CTLs. We demonstrated that CD8 iTreg tumoricidal activity is dependent on the release of perforin-containing thrombospondin-4⁺ SMAPs. Additionally, the generation of dual functional (suppressor; cytolytic) CD8 iTreg expressing a CAR19-41BB receptor enhanced the targeted cytotoxicity of this cell population. We demonstrated that addition of CAR19-41BB to CD8 iTreg augments the targeted killing efficacy against CD19+ B-cell leukemia (Nalm-6) in vitro, while retaining in vitro suppressor capacity. In vivo, CAR19-41BB CD8 iTreg exhibit superior tumoricidal activity compared to CAR19-41BB CTLs. Additionally, using a model of xenoGVHD with residual leukemia, we demonstrate the CAR19-41BB are capable of simultaneous suppression of Nalm6 tumor burden and GVHD.

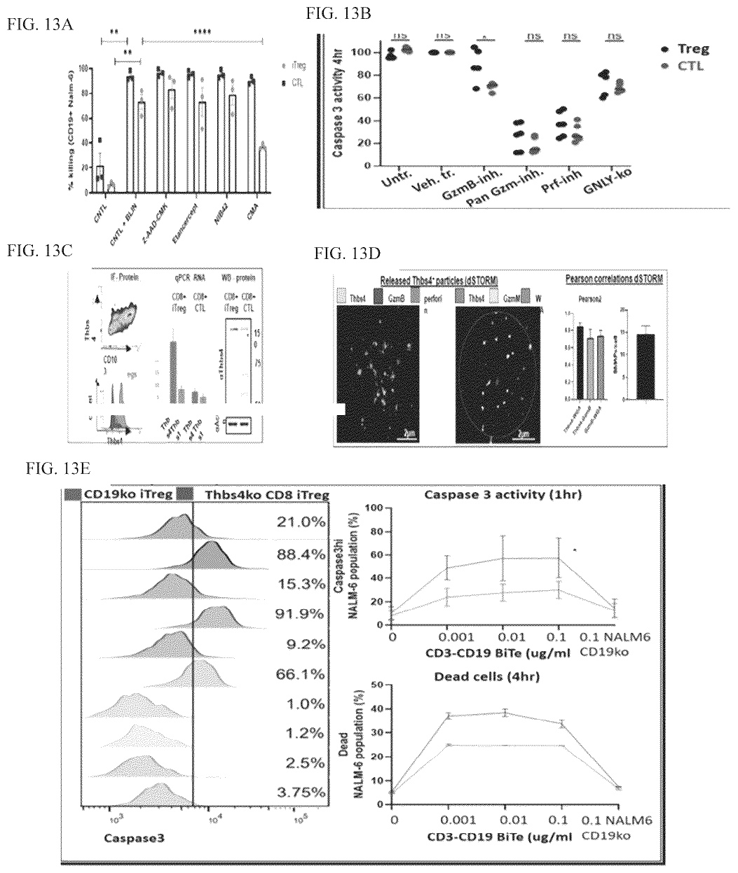

FIG. 13 shows that human CD8 iTreg are highly cytotoxic. FIGS. 13A and 13B demonstrates that CD8 iTreg killing is dependent on the release of perforin and granzymes, although not specific to granzyme B. FIGS. 13C and 13D demonstrate that, unique from CD8 CTLs, CD8 iTreg release thbs4+ SMAPs partials, which, FIG. 13E shows are required for effective tumoricidal activity in vitro.

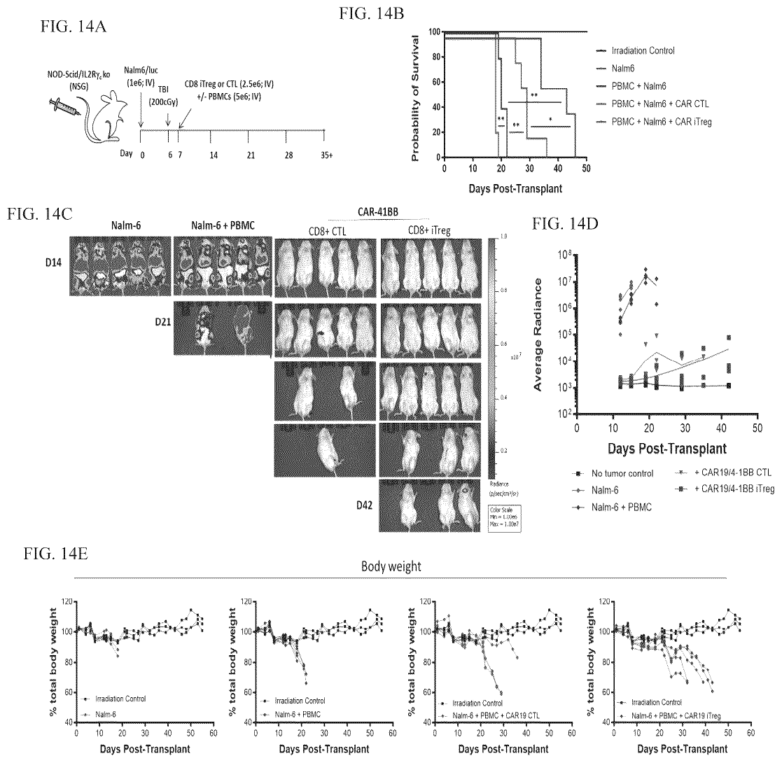

CAR19-41BB CD8 simultaneously reduced GVHD severity, indicated by delayed weight loss, and reduced tumor burden in a xenogeneic GVHD residual tumor model (FIG. 14). Treatment with CAR19+ CTLs produced robust antileukemic activity, protecting mice from fatal tumor progression (FIG. 14B-14D). However, CD19+ CAR CTL recipients exhibited no protection against GVHD, resulting in significant early weight loss in those animals (FIG. 14E) and only a marginal improvement in median survival compared to Nalm-6/PBMC recipients (29- vs. 20-days, respectively; p<0.0026; FIG. 14B). Conversely, mice receiving CAR19 iTreg intervention had reduced early weight loss (FIG. 14E), blunted leukemia progression (FIGS. 14C and 14D), and significantly prolonged animal survival compared to CAR19 CTL recipients (43-day median survival; p<0.0173; FIG. 14B).

Analysis of CD8 iTreg are performed to identify populations of CD8 iTreg with optimal T-cell suppression and tumor killing characteristics. The application of dual functional CD8 iTreg is expanded to additional models of blood cancer, including VISTA-, CD33-, CD123-, CD45- and CD83-CAR targets for the treatment of AML by CD8+ iTreg. Analysis of the bifunctional properties of these cells is continued in xenogeneic mouse models of GVHD and leukemia tumor clearance; and metabolic and functional analysis of CD8 iTreg, including expanded analysis of major mechanisms of suppression, cytotoxic killing pathways including SMAPs analysis is completed.

### Example 4—Characterization of CD103+ CD8+ CD8 iTreg SMAPs Mediated Cytotoxicity

Studies were initiated to determine the mode-of-action of CD103+ CD8 iTreg cytolytic function, focusing on supramolecular attack particles (SMAPs). Studies were initiated to characterize their secretome and their method of efficiently eliminating NALM-6 leukemia cells. The effects of CAR19-4-1BB transduced CD8 iTreg on antigen-specific tumor cytotoxicity and suppression was evaluated and compared to their CD8+ CTL counterparts.

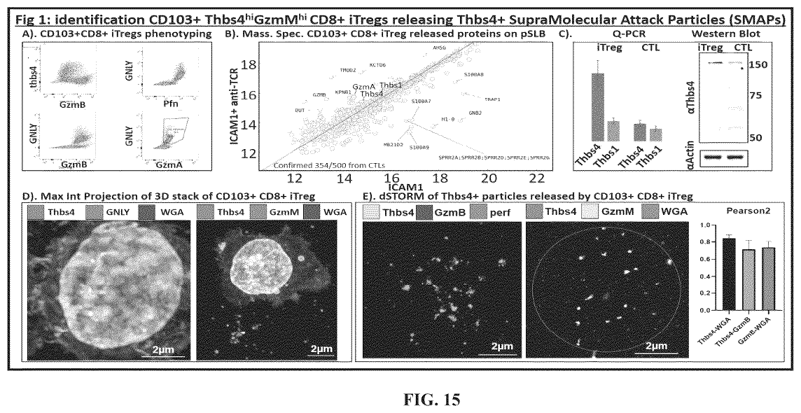

Cytotoxic CD103+ CD8+ iTregs were identified (FIG. 15). Studies were performed to identify the most cytotoxic population of CD8 iTregs. The CD103+ population of CD8 iTregs have a population that express high Thbs-4, tumoricidal protein Granulolysin (GNLY), and multiple Granzymes including GzmA and GzmM (15A). Mass spectrometry identified 557 proteins released by CD103+CD8+ iTregs on TCR-activating planar supported lipid bilayers (pSLBs) including Thbs's and Gzm's, with extensive overlap (354/500) of proteins released by CTLs under similar conditions (15B). Notably, no pro-inflammatory cytokines were released in Treg secretome such as IFNgamma that was identified in CTL secretome. We previously identified particles released by CTLs containing granzyme B and perforin inside a carbon-dense shell containing Thbs1 and WGA-stained highly glycosylated proteins. We named these cytotoxic particles SupraMolecular Attack Particles (SMAPs). Surprisingly, we found that CD8 iTregs preferentially express Thbs4 rather than Thbs1 (15C) that can be found intracellularly or trailing as released particles behind the Treg with cytotoxic cargo (15D), including GNLY and GzmM. After release and flushing off CD8+ iTregs, the particles left behind can be analysed with super-resolution microscopy dSTORM and similarly confirm presence of endogenously expressed Thbs4, GzmMs, Perforin, and GNLY. Building on our initial description of CTL-derived SMAPs with cryo-soft X-ray tomography, we transiently expressed GzmB-mCherry-pHluorin in CD8 iTregs and analyzed their particles released on TCR-activating EM-grids with fluorescent 3D cryo-SIM correlative with cryo-soft X-ray tomography. Green fluorescence demonstrate pH-sensitive GzmB-mCherry-pHluorin is away from quenching acidic pH present in lytic granules, demonstrating released GzmB. This released GzmB resides at the core of a carbon-dense shell similar to the CTL-derived SMAPs.

CD103+CD8+ iTreg mode-of-action was evaluated (FIG. 13D-13F). Granzymes induce cell death by caspase 3. Using this upstream target, we assessed NALM-6 leukemia cell killing by CD103+CD8+ iTreg upon different inhibitor treatments (13D) and demonstrated that Treg and CTLs rely on perforin for CD3-CD19 bispecific engager (BiTE)-induced killing. Interestingly, Tregs cytotoxicity was less affected by GzmB-specific inhibition but similarly sensitive to pan-Granzyme protease inhibitors as CTLs. This suggests CTLs rely heavily on Granzyme B whereas CD103+CD8+ iTregs use a more diverse repertoire of Granzymes for killing NALM-6 tumor cells. Knocking out Thbs4 expression in most of CD103+CD8+ iTregs resulted in 50% decrease in killing NALM-6 leukemia cells in vitro detected after 4 hours or as caspase 3 activity after the first 60 minutes of co-incubation (19B). Co-incubating CD19+ NALM-6 tumor cells with pSLBs containing released content and SMAPs (see FIG. 15A) was able to induce caspase-3 mediated cell death relying on Thbs4 expression (13C). The cytotoxic potential of SMAPs released by CTLs is inferior to the SMAPs from CD103+CD8+ iTregs.

Generating CAR19-41BB CD8 iTreg amplified antigen specific cytotoxicity without compromising T-cell suppression (FIG. 8). Expression of chimeric antigen receptors (CAR) reactive against tumor cells has proved to be highly effective in treating B cell malignancies. However, such immunotherapy with CD8 CTLs can lead to complications such as cytokine release syndrome and immune-effector cell-associated neurotoxicity syndrome. In contrast, immune suppressive CD8 iTregs could blunt these side-effects. Moreover, for patients who relapse after allogeneic hematopoietic stem and progenitor cell transplantation, CD8 CTLs would have a risk for graft-versus-host disease, a potentially lethal complication. Therefore, we transduced CD8 CTLs and CD8 iTregs with a CAR19-4-1BB lentivirus, selected transductants, and tested for antigen-specific leukemia cell killing in vitro. CAR19-4-1BB CD8 iTregs augmented antigen-specific tumor cell killing function without compromising suppressor function.

In a series of studies examining CD8 iTreg subsets by distinguishing phenotypic characterization, we found that the CD103+ subset was highly suppressive. Moreover, this subset also was highly cytolytic, which we traced to perforin expression in part and identified supramolecular attack particles containing thrombospondin-4 (Thb4), granzymes and perforin that had been secreted and are capable of target cell killing. Transduction of CD8 iTregs with a CD19scFv-4-1BB chimeric antigen receptor augmented their cytolytic function that resulted in superior in vitro anti-tumor function without abrogating suppressor function.

GzmB-mCherry-pHluorin is transiently expressed in CD8 iTregs and the particles released are analyze on TCR-activating EM-grids with fluorescent 3D cryo-SIM correlative with cryo-soft X-ray tomography. pH-sensitive GzmB-mCherry-pHluorin are quantified by green fluorescence as a means of demonstrating released cytolytic molecules, their location in SMAPs is determined and those data are compared to CD8 CTLs. The effects of signaling through the CAR by CD19 in driving the release of SMAPs are tested and CAR compared to TCR in triggering SMAP release donor-matched CD103+CD8+ iTregs. dSTORM is used to assess whether the release of Thbs4+ SMAPs is similarly triggered by CAR19 compared to TCR signaling. Whether continuous CD19-mediated triggering of CAR19-CTLs and CAR19−CD8+ Tregs impairs Nalm-6 killing by CTLs more than Tregs and causes increased expression of exhaustion antigens is determined. CAR19-41BB CD8 iTreg and CAR19-41BB CD8 CTLs are tested for clearing CD19+ Nalm-6 tumor in vivo. If CAR19-41BB CD8 iTregs are effective in clearing Nalm-6 in vivo, whether CAR19-41BB CD8 iTregs can mediate a graft-versus-leukemia effect and simultaneously suppress graft-versus-host disease is determined.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of increasing the cytolytic and/or anti-tumor function of CD8 iTregs, comprising:
transducing the CD8 iTregs with a construct, wherein the construct expresses a ligand, thereby producing ligand-expressing CD8 iTregs,
thereby increasing the cytolytic and/or anti-tumor function of the ligand-expressing CD8 iTregs compared to CD8 iTregs not containing or expressing the ligand.

2. The method of claim 1, wherein the transduction does not abrogate suppressor function of the CD8 iTregs.

3. The method of claim 1, wherein the construct expressing a ligand is a CAR construct.

4. The method of claim 1, wherein the construct expresses a ligand selected from CD19, CD33, CD123, CD45, CD83, and VISTA.

5. The method of claim 1, wherein the ligand expressed by the construct is a ligand that specifically recognizes a pathogenic antigen, a tumor antigen, a foreign antigen, or a self-antigen.

6. The method of claim 1, wherein the ligand-expressing iTreg exhibit cytotoxicity and suppressor function.

7. The method of claim 1, wherein the ligand-expressing iTregs are cytotoxic to tumor cells.

8. A method of delivering therapy to a patient in need thereof, comprising:
providing ligand-expressing CD8 iTregs;
introducing the ligand-expressing CD8 iTregs into the patient in need thereof, thereby delivering therapy to a patient in need thereof.

9. The method of claim 8, wherein the CD8 Tregs used to produce the ligand-expressing CD8 iTregs are obtained from the patient.

10. The method of claim 8, wherein the method does not suppress graft-vs-tumor (GVT) activity in the patient.

11. The method of claim 8, wherein the method augments graft-vs-tumor (GVT) activity in the patient.

12. The method of claim 8, wherein the method reduces or eliminates tumor cells in the patient.

13. The method of claim 8, wherein the patient has undergone a hematopoietic stem cell transplantation (HSCT).
